(19) United States
(12) Patent Application Publication
(10) Pub. No.: US 2025/0002468 A1
(43) Pub. Date: Jan. 2, 2025

(54) EPOXIDATION OF OLEFIN MIXTURE

(71) Applicant: TEKNOLOGIAN TUTKIMUSKESKUS VTT OY, Espoo (FI)

(72) Inventors: Sari RAUTIAINEN, Espoo (FI); Juha LEHTONEN, Espoo (FI); Tapio SALMI, Turku (FI); Matias ALVEAR, Turku (FI); Michele Emanuele FORTUNATO, Santa Maria La Carita (Napoli) (IT)

(21) Appl. No.: 18/708,732

(22) PCT Filed: Nov. 3, 2022
(86) PCT No.: PCT/FI2022/050721

§ 371 Date: May 9, 2024

(30) Foreign Application Priority Data
Nov. 16, 2021 (FI) 20216175

(51) Int. Cl. C07D 301/12; B01J 21/06; C07C 1/12

(52) U.S. Cl. CPC C07D 301/12; B01J 21/063; C07C 1/12

(57) ABSTRACT

The present disclosure relates to a process for epoxidizing an olefin mixture (11), comprising at least two olefins selected from the group consisting of ethylene, propylene and 1-butylene. Particularly the present disclosure relates to a process comprising contacting the olefin mixture (11) and solvent with hydrogen peroxide (12) in the presence of a heterogeneous Lewis acid catalyst in a reactor; and recovering a product mixture (15) corresponding to the provided olefin mixture (11). The present disclosure further concerns the product obtained by the method as well as the use of the recovered product mixture in the production of polycarbonates, cyclic carbonates and/or polyols, preferably polycarbonate or polyether polyols for use in polyurethane products. The present disclosure also relates to an integrated process for producing epoxides, wherein the olefin mixture is separated from Fischer-Tropsch synthesis (7).

### FIELD OF THE DISCLOSURE

The present disclosure relates to a process for epoxidizing an olefin mixture, comprising at least two olefins selected from the group consisting of ethylene, propylene and 1-butylene. Particularly the present disclosure relates to a process comprising contacting the olefin mixture and solvent with hydrogen peroxide in the presence of a heterogeneous Lewis acid catalyst in a reactor; and recovering a product mixture corresponding to the provided olefin mixture. The present disclosure further concerns the product obtained by the method as well as the use of the recovered product mixture in the production of polycarbonates, cyclic carbonates, polyether polyols and/or polycarbonate polyols and a polyurethane product comprising the abovementioned polyols. The present disclosure also relates to an integrated process for converting olefins, having carbon number 2 to 4, to epoxides, wherein the olefin mixture is separated from Fischer-Tropsch synthesis. The raw materials for the Fischer-Tropsch synthesis typically originate from sustainable sources as captured CO₂ and green hydrogen or from biomass and wastes gasification.

### BACKGROUND OF THE DISCLOSURE

Generally, epoxides are formed by the reaction of an olefin with an oxidizing agent in the presence of a catalyst. Many different methods for the preparation of epoxides have been developed.

One commercially practiced technology is producing ethylene oxide by direct oxidation with oxygen in a silver catalysed gas phase process. Propylene oxide and 1-2 epoxybutane are typically produced commercially using oxidation by organic peroxides, such as tert-butyl hydroperoxide or cumene hydroperoxide. Another oxidizing agent useful for the preparation of epoxides is hydrogen peroxide. For example, documents WO 2010/036296 and WO 2009/108264 discloses catalysts useful in olefin epoxidation with hydrogen peroxide for epoxidation of an olefin such as propylene.

US 2009/131693 discloses a process for selective oxidation of propylene to propylene oxide or ethylene to ethylene oxide, comprising the step of contacting the olefin with an oxidant in the presence of a catalyst and organic base, in a solvent system comprising an organic solvent.

Despite the ongoing developments in the field, there is still a need for further improvements in the epoxidation methods.

### BRIEF DESCRIPTION OF THE DISCLOSURE

An object of the present disclosure is to provide a process for epoxidizing an olefin mixture, instead of first separating olefins of different length from each other.

The object of the disclosure is achieved by the process, the product obtainable by the method and the use of the recovered product mixture, which are characterized by what is stated in the independent claims. The preferred embodiments of the disclosure are disclosed in the dependent claims.

The disclosure is based on the idea that an olefin mixture, comprising at least two olefins selected from the group consisting of ethylene, propylene and 1-butylene, is epoxidized simultaneously with high conversion to a product mixture corresponding to the provided olefin mixture. By performing mixed olefins epoxidation, expensive fractionation of 1-olefins can be avoided. Typically, separation of olefins with only one carbon difference is energy consuming and requires very tall and expensive distillation columns.

An advantage of process of the disclosure is that the epoxidation process is a non-hazardous, environmentally friendly process, with water as the only stocihiometric by-product. Because the inventive process uses a liquid oxidant (e.g., hydrogen peroxide), oxygen in the vapor phase can be avoided. Thus, many of the challenges of state of the art processes can be avoided, for example the formation explosive mixture of O₂/ethylene oxide which is formed in the direct epoxidation of ethylene to ethylene oxide by reaction with oxygen over a silver catalyst and the problems of the formation of by-product alcohol from the organic peroxides.

A further advantage of the use of hydrogen peroxide as oxidant is that it is cheaper than organic peroxides, since organic peroxides are commonly produced from hydrogen peroxide.

Advantageously, by the process of the disclosure, high selectivity, over 90%, preferably over 95% is obtained in the epoxidation of an olefin mixture comprising at least two olefins selected from the group consisting of ethylene, propylene and 1-butylene. Advantageously, the conversion of the olefins of the olefin mixture is as good when epoxidized in a mixture compared to when the olefins are pure compounds. The mixture of epoxides can for example be used as such for polyol, polycarbonate or cyclic carbonates synthesis.

A further advantage of the process of the disclosure is that it enables utilization of mixed olefins from Fischer-Tropsch synthesis using syngas originating e.g. from CO₂ and green hydrogen or renewable resources that can be further used for producing valuable specialty chemicals and/or polymers, enabling a carbon neutral economy.

Further advantages are the effective utilization of the raw-material, for example of mixed olefins from Fischer-Tropsch synthesis, as well as the reduction of the environmental footprint related to the use of captured carbon dioxide in the process. The utilization of the raw material is especially effective if unreacted olefins are recirculated back into the epoxidation process.

### BRIEF DESCRIPTION OF THE DRAWINGS

In the following the disclosure will be described in greater detail by means of preferred embodiments with reference to the accompanying drawings, in which

FIG. 1 is a schematic flow diagram representing one embodiment of the process;

FIG. 2 is a schematic flow diagram representing one embodiment of the process;

FIG. 3 shows conversion (%) and selectivity (%) of epoxidation of individual alkanes;

FIG. 4 shows temperature effect on conversion (%) and selectivity (%) in olefins mixture epoxidation;

FIG. 5 shows H₂O₂ concentration effect on conversion (%) and selectivity (%) in olefins mixture epoxidation;

FIG. 6 shows the effect of the mixture composition on selectivity (%); and

FIG. 7 shows the effect of the amount of catalyst on conversion (%) and selectivity (%).

### DETAILED DESCRIPTION OF THE DISCLOSURE

The disclosure relates to a process for epoxidizing an olefin mixture comprising: providing an olefin mixture, comprising at least two olefins selected from the group consisting of ethylene, propylene, 1-butylene, and a solvent chosen from the group consisting of light monohydric alcohols and non-protic organic solvents; contacting the olefin mixture and solvent with hydrogen peroxide in the presence of a heterogeneous Lewis acid catalyst in a reactor; and recovering a product mixture comprising at least two epoxides corresponding to the provided olefin mixture.

The disclosure further relates to the use of the recovered product mixture in the production of polyols, cyclic carbonates, polycarbonate polyols and/or polyether polyols as well as to a polyurethane product comprising the polyol. The recovered product mixture is further used in the production of other products where a mixture of at least two epoxides of the disclosure is suitable.

The disclosure also relates to a product obtainable by the embodiments of the process of the disclosure.

The term “1-olefins”, as used in the disclosure refers to olefins with a double bond in terminal position, including ethylene and propylene.

In embodiments of the disclosure the olefin mixture comprises at least two C₂-C₄ linear 1-olefins, i.e. at least two olefins selected from the group consisting of ethylene, propylene, 1-butylene. Typically, the olefin mixture comprises or consists of a mixture of ethylene and propylene; a mixture of propylene and 1-butylene; a mixture of ethylene and 1-butylene; or a mixture of ethylene, propylene and 1-butylene. Advantageously no other olefins are present in the reaction taking place in the reactor. Minor amounts of for example methane, longer olefins (+C₅) or alkanes can be present in the olefin mixture as impurities. Preferably the amount of impurities is below 10 wt %, preferably below 5 wt %. The olefin mixture of the process of the disclosure is typically recovered from Fischer-Tropsch synthesis, preferably the mixture is produced by iron catalysed Fischer-Tropsch synthesis. It is advantageous that the olefins of the olefin mixture are epoxidized directly together, without first separating and/or purifying olefins of the olefin mixture. In some embodiments of the disclosure, methane and/or unreacted feed are recirculated from the Fischer-Tropsch synthesis back to the reverse water-gas shift reactor combined with catalytic partial oxidation. The longer hydrocarbons (C₅+) obtained from the Fischer-Tropsch synthesis are advantageously used as fuel, fuel components or for production of fuels, fuel components or other chemicals than epoxides.

Preferred embodiments of the disclosure further comprise an integrated process for producing epoxides, wherein the process comprises the steps of reacting carbon dioxide and hydrogen in a reverse water-gas shift reactor and/or catalytic partial oxidation reactor, followed by separation of water, carbon monoxide and hydrogen. The carbon monoxide and hydrogen are directed into a reactor for Fischer-Tropsch synthesis where after olefins having carbon number 2 to 4, hydrocarbons having carbon number over 5, water as well as optionally methane and unreacted feed is separated after said Fischer-Tropsch synthesis. The olefins having carbon number 2 to 4 are directed to an epoxidation reactor and further to distillation to obtain a mixture of epoxides having carbon number 2 to 4 and optionally unreacted olefins. Optionally, the integrated process further comprises recirculating methane and unreacted feed from the Fischer-Tropsch synthesis reactor back to the reverse water-gas shift reactor/catalytic partial oxidation reactor and/or recirculating unreacted olefins from distillation back to the epoxidation, either to the olefins feed or to the epoxidation reactor. Typically, the olefins having carbon number 2 to 4 are an olefin mixture, comprising at least two olefins selected from the group consisting of ethylene, propylene and 1-butylene.

The recovered product mixture corresponds to the provided olefin mixture, meaning that a mixture of epoxides (ethylene oxide, propylene oxide and/or 1,2-epoxybutane) corresponding to the olefin mixture, i.e. the fed 1-olefins is obtained as a product. The epoxidation of the different olefins is performed simultaneously. For example, a mixture of ethylene and propylene can be epoxidized simultaneously with high conversion to a mixture of ethylene oxide and propylene oxide. Typically, the process of the disclosure comprises separating the product mixture from unreacted alkenes (olefins), hydrogen peroxide, solvent and/or possible by-products, by evaporation, distillation and/or liquid-liquid extraction. The product mixture or the product, i.e. the mixed epoxides separated from unreacted olefins, hydrogen peroxide, solvent and/or by-products can be used as such for polyol, cyclic carbonate and/or polycarbonate production using conventional processes. The polyols are typically utilized in polyurethane applications. In some embodiments of the disclosure unreacted olefins, separated from the product mixture of epoxides, are recirculated back to epoxidation as part of the olefin mixture. The possible by-products are typically formed from reactions between solvent and olefins.

In embodiments of the disclosure the solvent is chosen from the group of light monohydric alcohols, preferably methanol, ethanol or propanol or non-protic organic solvents such as acetonitrile.

Hydrogen peroxide is used as oxidant in epoxidation of the process of the disclosure. Typically, the hydrogen peroxide is fed to the reactor in the form of a water solution. Typically, the concentration of hydrogen peroxide is 1-4 wt % of the total liquid feed in the reactor comprising the olefin mixture and solvent, preferably the concentration of hydrogen peroxide is 1.5-3 wt %, more preferably 1.5-2.5 wt %, most preferably about 2 wt %.

In embodiments of the disclosure the process is catalysed by heterogeneous Lewis acid catalysts. Typically, the heterogeneous Lewis acid catalysts is silicate doped with titanium, niobium, molybdenum and/or tungsten, preferably titano-silicates or niobium incorporated mesoporous silicates, more preferably TS-1 titano-silicates. The catalyst is preferably located in the reactor as a fixed bed, but also other state of the art set ups are possible. Typically, the weight hourly space velocity (WHSV) based on olefins feed rate to the reactor, i.e. based on the feed rate of the olefin mixture, is 0.1-2 1/hr, preferably the WHSV is 0.3-1 1/hr, more preferably 0.4-0.9 1/hr.

In embodiments of the disclosure the temperature range were epoxidation is performed is typically 15-75° C., preferably 25-55° C., more preferably 40-50° C., including the temperature being a temperature between two of the following temperatures; 15° C., 20° C., 25° C., 30° C., 35° C., 40° C., 45° C., 50° C., 55° C., 60° C., 65° C., 70° C. and 75° C. The pressure range were epoxidation is performed is typically 0.5-10 bar, preferably 0.5-5 bar, including the pressure being a pressure between two of the following pressures; 0.5 bar, 1 bar, 1.5 bar, 2 bar, 3 bar, 4 bar, 5 bar, 6 bar, 7 bar, 8 bar, 9 bar and 10 bar.

The processes of this invention may be performed in batch mode, semi-batch mode, or continuous mode. Preferably in continuous mode.

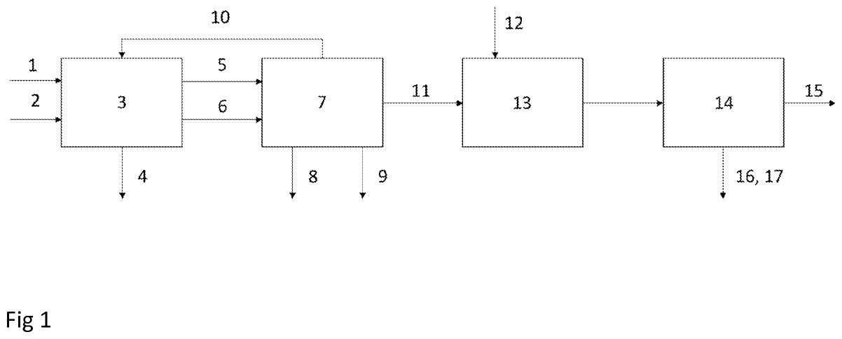

FIG. 1 shows an embodiment of the disclosure where captured carbon dioxide 1 and hydrogen 2 are reacted in a reverse water-gas shift/catalytic partial oxidation reactor 3. Water 4 is separated and the obtained carbon monoxide 5 and hydrogen 6 are fed into a reactor for Fischer-Tropsch synthesis 7. Methane and unreacted feed 10 are optionally recirculated from the Fischer-Tropsch synthesis reactor 7 back to the reverse water-gas shift/catalytic partial oxidation reactor 3. From the Fischer-Tropsch synthesis longer C₅+ hydrocarbons 8 and water 9 are separated and the obtained C₂-C₄ olefins 11 are fed to an epoxidation reactor 13 together with hydrogen peroxide 12 and the reaction product mixture is directed further to distillation 14. C2-C4 epoxides 15 are separated as well as optionally also unreacted olefins 16 and/or by-products 17.

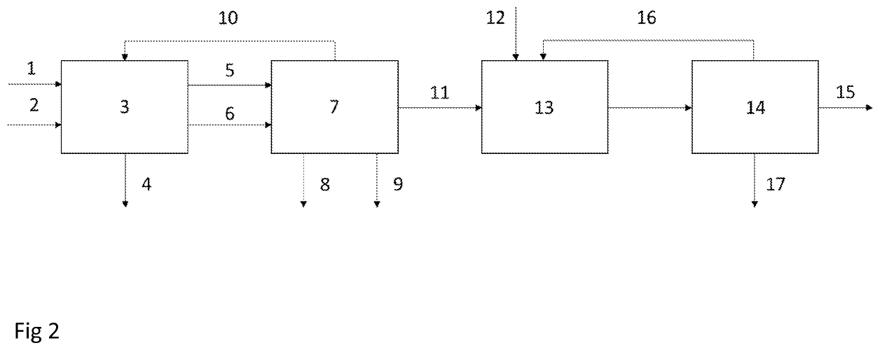

FIG. 2 shows an embodiment of the disclosure where captured carbon dioxide 1 and hydrogen 2 are reacted in a reverse water-gas shift reactor 3. Water 4 is separated and the obtained carbon monoxide 5 and hydrogen 6 are fed into a reactor for Fischer-Tropsch synthesis 7. Methane and unreacted feed 10 are optionally recirculated from the Fischer-Tropsch synthesis reactor 7 back to the reverse water-gas shift reactor 3. From the Fischer-Tropsch synthesis longer C₅+ hydrocarbons 8 and water 9 are separated and the obtained C₂-C₄ olefins 11 are fed to an epoxidation reactor 13 together with hydrogen peroxide 12 and the reaction product mixture is directed further to distillation 14. C2-C4 epoxides 15 and optionally also by-products 17 are separated. Unreacted olefins 16 are optionally recirculated from distillation 14 to epoxidation reactor 13 or to the olefins feed (not shown).

### EXAMPLES

### Comparative Example 1 Epoxidation of Individual Olefins

Epoxidation of three individual olefins, ethylene, propylene and 1-butene was performed. The respective olefin and nitrogen were fed into the reactor for approximately 2 hours for catalyst TS-1 (1 g) pre-saturation. After the steady state of the gas phase had been reached, the liquid was fed in too. The liquid feed solution was prepared by mixing 35 wt % hydrogen peroxide in water-with methanol. The temperature used was 45° C., the pressure 1 bar and the liquid flow 0.5 mL/min. The hydrogen peroxide concentration in the liquid feed was 2 wt %. The ethylene, propylene or 1-butene molar flow was 0.223 mmol/min respectively. The total gas flow (N2+C4H8) or (N2+C3H6) or (N2+C2H4) was 0.446 mmol/min.

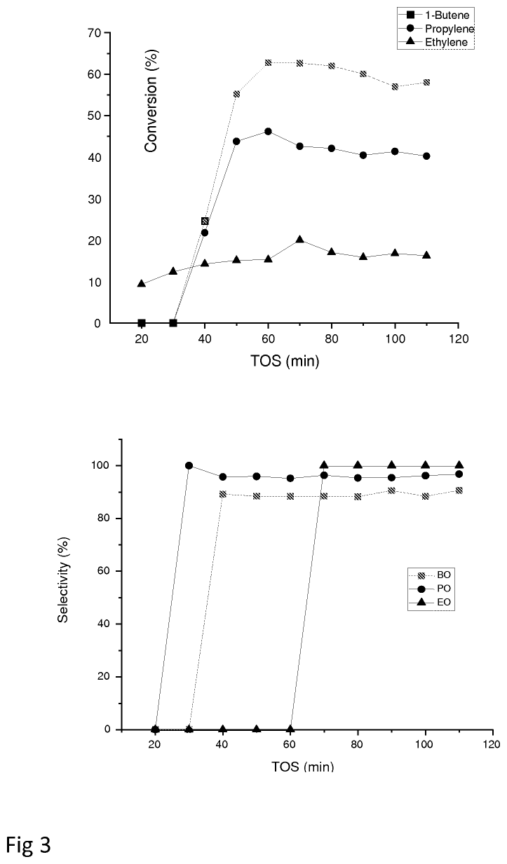

The conversion and selectivity of respectively olefin are shown in FIG. 3, showing high selectivity. However, a drawback of the process is that the olefins need to be separated from each other before epoxidation.

### Example 2 Light Olefins Mixture Epoxidation

Catalyst TS-1 (1 g) pre-saturation was performed. The mixtures of olefins and nitrogen were fed into the reactor for approximately 2 h. After the steady state of the gas phase had been reached, the liquid was fed in too. The liquid feed solution was prepared by mixing 35 wt % hydrogen peroxide in water with methanol.

Different hydrogen peroxide concentrations (1-4 wt %), temperatures (15-55° C.) and different mixture compositions were investigated.

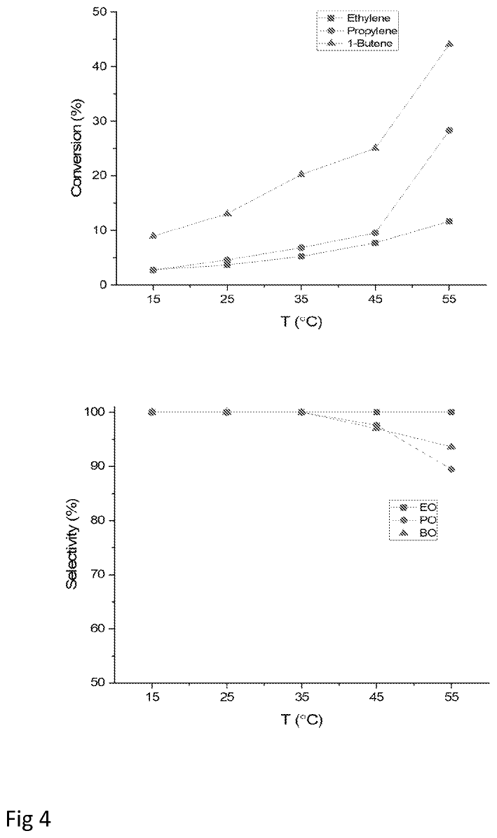

The results are shown in FIGS. 4-6.

The effect of temperature on the conversion and selectivity of the olefins of the mixture was studied at 15° C., 25° C., 35° C., 45° C. and 55° C. The pressure was 1 bar, the amount of hydrogen peroxide was 2 wt %, the ethylene, propylene and 1-butene molar flows were maintained at 0.116 mmol/min respectively and the liquid flow was 0.5 mL/min. The total gas flow (N₂+C₄H₈+C₃H₆+C₂H₄) was 0.446 mmol/min. The results are shown in FIG. 4. The maximum conversion was observed at ca. 55° C. for all olefins, while the highest selectivity to epoxide was achieved at 15° C., 25° C. and 35° C. However, the selectivity was over 85% for all temperatures.

The influence of hydrogen peroxide was studied at 45° C. and 1 bar when the ethylene, propylene and 1-butene molar flows were maintained at 0.116 mmol/min respectively and the liquid flow was 0.5 mL/min. The total gas flow (N₂+C₄H₈+C₃H₆+C₂H₄) was 0.446 mmol/min. The amount of hydrogen peroxide was 1 wt %, 1.5 wt %, 2 wt % and 4 wt % of the liquid feed.

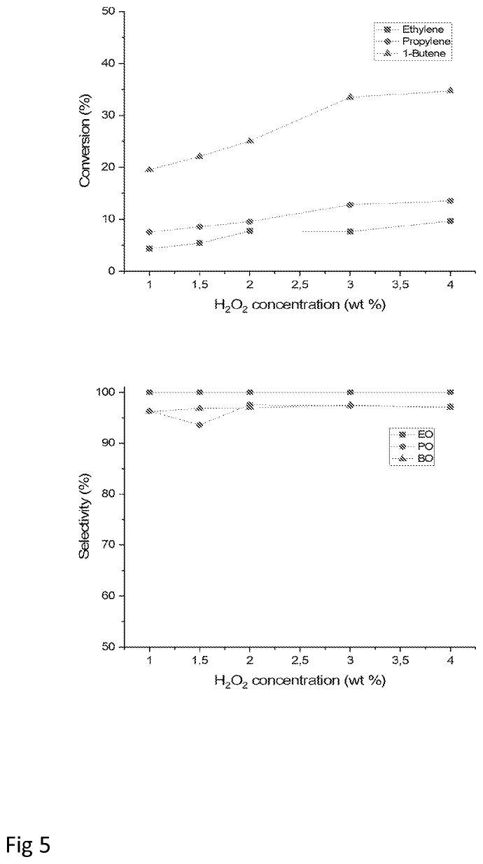

FIG. 5 shows the influence of hydrogen peroxide on conversion and selectivity. The 4 wt % amount of hydrogen peroxide resulted in the highest conversion, while the selectivity to the oxides was highest for 2 wt %, 3 wt % and 4 wt %. The change in the conversion and selectivity was clearly noticeable between 2 wt % and 3 wt % hydrogen peroxide for 1-butene and propylene.

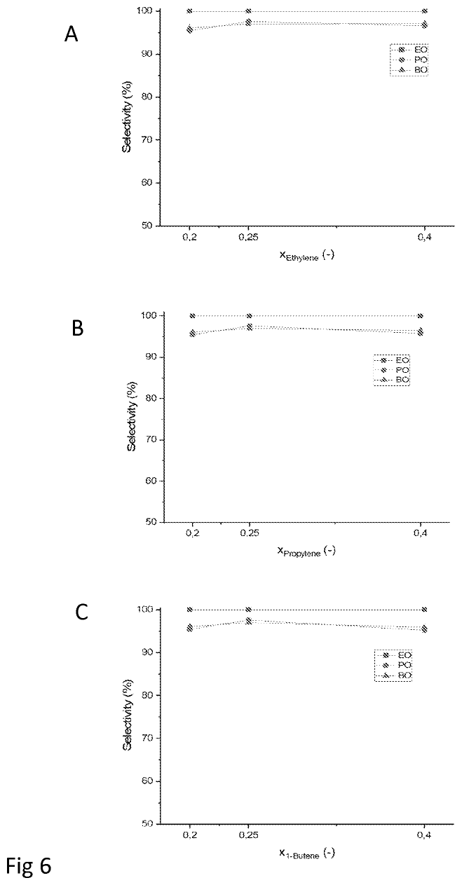

The effect of 1-olefin mixture composition on the selectivity of the olefins of the mixture are shown in FIG. 6. The influence of the composition of the mixture of 1-olefins was studied at 45° C. and 1 bar when the ethylene, propylene and 1-butene fraction was varied between 0.2 and 0.4 respectively. The liquid flow was at 0.5 mL/min, the amount of hydrogen peroxide was 2 wt % and the total gas flow (N₂+C₄H₈+C₃H₆+C₂H₄) was 0.446 mmol/min. The ethylene, propylene and 1-butene fractions used are shown in Table 1-3.

TABLE 1

| X_{Ethylene} | X_{Propylene} | X_{1-Butene} | X_{N2} |
|---|---|---|---|
| 0.25 | 0.25 | 0.25 | 0.25 |
| 0.2 | 0.2 | 0.2 | 0.4 |
| 0.4 | 0.2 | 0.2 | 0.2 |

TABLE 2

| X_{Ethylene} | X_{Propylene} | X_{1-Butene} | X_{N2} |
|---|---|---|---|
| 0.25 | 0.25 | 0.25 | 0.25 |
| 0.2 | 0.2 | 0.2 | 0.4 |
| 0.2 | 0.4 | 0.2 | 0.2 |

TABLE 3

| X_{Ethylene} | X_{Propylene} | X_{1-Butene} | X_{N2} |
|---|---|---|---|
| 0.25 | 0.25 | 0.25 | 0.25 |
| 0.2 | 0.2 | 0.2 | 0.4 |
| 0.2 | 0.2 | 0.4 | 0.2 |

FIG. 6 A corresponds to Table 1, FIG. 6 B corresponds to Table 2 and FIG. 6 C corresponds to Table 3. As can be seen from FIGS. 6 A-C, the composition of the 1-olefin mixture did not have an effect on the conversion and selectivity.

The effect of the amount of catalyst on conversion and selectivity was studied at 45° C. and 1 bar. The amount of hydrogen peroxide was 2 wt % and the ethylene, propylene and 1-butene molar flows were maintained at 0.116 mmol/min respectively and the liquid flow was 0.5 mL/min. The total gas flow (N₂+C₄H₈+C₃H₆+C₂H₄) was 0.446 mmol/min. The catalyst amount in the experiments was varied from 1 g to 2 g, so the weight hourly space velocity (WHSV) based on the feed rate of the olefin mixture to the reactor was 0.88 1/hr for 1 g catalyst and 0.44 1/hr for 2 g catalyst.

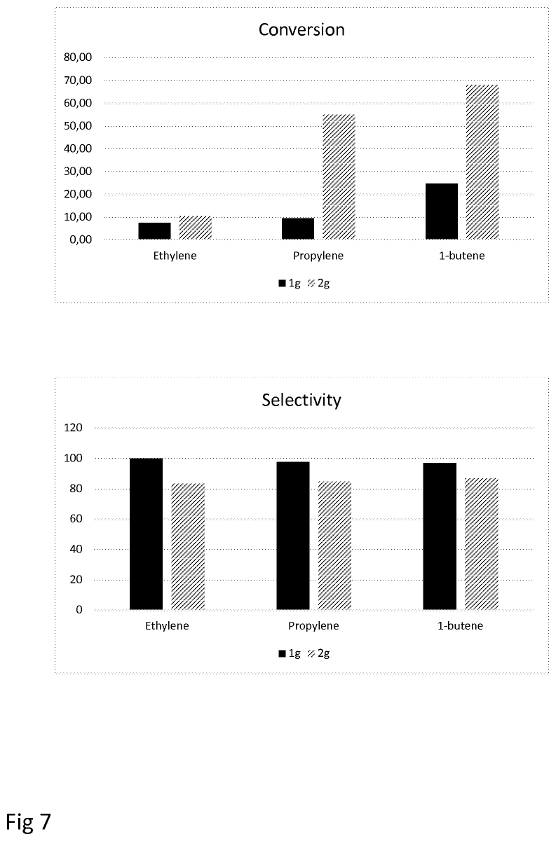

FIG. 7 shows the influence of catalyst amount on the selectivity and conversion. The higher catalyst (2 g) amount resulted in significantly higher conversions of all olefins. However, the selectivity to epoxides was somewhat lower with 2 g of catalyst.

1. A process for epoxidizing an olefin mixture, characterized in that the process comprises:
providing an olefin mixture, comprising at least two olefins selected from the group consisting of ethylene, propylene and 1-butylene, and a solvent chosen from the group consisting of water, light monohydric alcohols and non-protic organic solvents;
contacting the olefin mixture and solvent with hydrogen peroxide in the presence of a heterogeneous Lewis acid catalyst in a reactor; and
recovering a product mixture comprising at least two epoxides corresponding to the provided olefin mixture.

2. The process according to claim 1, characterized in that the heterogeneous Lewis acid catalyst is silicate doped with titanium, niobium, molybdenum and/or tungsten, preferably titano-silicates or niobium incorporated mesoporous silicates.

3. The process according to claim 1, characterized in that the light monohydric alcohol is methanol, ethanol or propanol.

4. The process according to claim 1, characterized in that the non-protic organic solvents is acetonitrile.

5. The process according to claim 1, characterized in that the reactor has a temperature between 15-75° C., preferably 25-55° C., more preferably 40-50° C.

6. The process according to claim 1, characterized in that the hydroxide peroxide concentration in the liquid feed is 1-4 wt %, preferably 1.5-3 wt %, more preferably 1.5-2.5 wt %, most preferably about 2 wt %.

7. The process according to claim 1, characterized in that the process further comprises recovering the olefin mixture from Fischer-Tropsch synthesis.

8. The process according to claim 1, characterized in that the catalyst is preferably located in the reactor as a fixed bed.

9. The process according to claim 1, characterized in that the weight hourly space velocity (WHSV) based on feed rate of the olefin mixture to the reactor is 0.1-2 1/hr, preferably the WHSV is 0.3-1 1/hr, more preferably 0.4-0.9 1/hr.

10. The process according to claim 1, characterized in that the process further comprises separating the product mixture from unreacted olefins, by-products formed in epoxidation, hydrogen peroxide and/or the solvent by evaporation, distillation and/or liquid-liquid extraction.

11. The process according to claim 10, characterized in that the process further comprises recirculation of unreacted olefins to the olefin mixture to be fed to epoxidation reactor.

12-16. (canceled)

17. The process according to claim 7 characterized in that said olefin mixture is recovered by
reacting carbon dioxide and hydrogen in a reverse water-gas shift reactor and/or catalytic partial oxidation reactor,
followed by separation of water, carbon monoxide and hydrogen,
directing said carbon monoxide and hydrogen into a reactor for Fischer-Tropsch synthesis,
separating olefins having carbon number 2 to 4, hydrocarbons having carbon number over 5, water and optionally methane and unreacted feed from said Fischer-Tropsch synthesis and said olefins having carbon number 2 to 4 are directed to an epoxidation reactor and further to distillation to obtain a mixture of epoxides having carbon number 2 to 4 and optionally unreacted olefins, wherein said olefins having carbon number 2 to 4 comprises at least two olefins selected from the group consisting of ethylene, propylene and 1-butylene.

18. The process according to claim 17 characterized in that said process further comprises:
recirculating methane and unreacted feed from the Fischer-Tropsch synthesis reactor back to the reverse water-gas shift reactor and/or catalytic partial oxidation reactor, and/or
recirculating unreacted olefins from distillation to epoxidation.
